(19) United States
(12) Patent Application Publication
(10) Pub. No.: US 2005/0249709 A1
(43) Pub. Date: Nov. 10, 2005

(54) Bone substitute

(76) Inventors: Erhardt Wrabetz, Hochheim (DE); Michael Rode, Oberursel (DE); Thierry Stoll, Sutz (CH); Stephan Becker, Wien (AT); Ingo Wilke, Friedland (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/088,784

(22) Filed: Mar. 25, 2005

(30) Foreign Application Priority Data
Mar. 30, 2004 (DE) 102004016065.1

(51) Int. Cl. A61K 45/00; A61F 2/00

(52) U.S. Cl. 424/93.7; 424/423

(57) ABSTRACT

A bone substitute which consists of a porous metallic or a biocompatible, open-cell material which is wholly or partly impregnated with a solution comprising factor XIII, or at least some of its pores are filled with a solution comprising factor XIII, is described.

The invention relates to a bone substitute consisting of a porous metallic or a biocompatible, open-cell material.

International patent application WO 02/15950 has disclosed a method for producing a bone substitute material in which a biocompatible, open-cell body is exposed to a vacuum, and osteoinductive and/or osteogenic substances in free-flowing form are sucked, by means of the vacuum generated in the pores of the body, into these pores. This makes it possible to produce a bone substitute material which comprises, in the pores of the biocompatible body, osteoinductive and/or osteogenic substances, which serves as network structure for new bone cells growing into the porous body.

This known method considerably improves the previously used methods for bone regeneration. However, there is a desire for bone regeneration to be even further simplified and expedited.

It has additionally been disclosed that coagulation factor XIII on systemic administration also, besides its wound-healing effect, exerts beneficial effects on the early callus formation phase and the late callus maturation phase. There is in this case a significant increase in the mechanical load-bearing capacity of the callus. The bone healing results are closely correlated with the chosen dose. The optimal dose has been found to be 10 and 50 U/kg. The beneficial results are attributable to the facts that, on the one hand, factor XIII quantitatively stimulates callus formation, presumably through the mitogenic effect on the osteoblasts, and, on the other hand, callus formation is faster owing to the quicker fibrin crosslinking in the hematoma. The increased rate of fibrin crosslinking can create favorable conditions for bone regeneration in the callus at an earlier time. Thus, giving factor XIII reduces the very long treatment times when callus formation and maturation are impaired, for example in cases of pseudarthroses or callus distractions. In addition, the rate of complications can also be reduced. Impregnation of a bone substitute with factor XIII and the special advantages, deriving therefrom, for bone regeneration have, by contrast, not previously been described.

Numerous experiments by scientific research groups have confirmed that factor XIII is able to expedite an improved bone healing. The question which therefore arose was whether a combination of the method disclosed in the international patent application WO 02/15950 for producing a bone substitute material with simultaneous administration of factor XIII is able to expedite bone regeneration. Important treatment methods in this connection consist both of the insertion of a bone implant and of in vivo or in vitro treatment of the bone material.

The basic requirements for successful anchoring of an implant with a porous surface include the use of a material with high biocompatibility, and optimization of the local surface conditions in the form of appropriate pore size, exclusion of relative movements at the implant/bone interface, and direct implant/bone contact. Implant manufacturers have to date mainly used metallic materials, employing pore sizes between 100μ and 500μ. Where it was possible in these experiments to investigate the effect of systemic administration of factor XIII concentrate and of recombinant factor XIII on bone ingrowth behavior and the firmness of anchoring of porous metallic surface implants, although a beneficial effect was evident, it could not be described as significant. It was not possible to infer from the experimental results disclosed to date whether the bone ingrowth behavior and the firmness of anchoring on the one hand, and the regeneration of bone material on a biocompatible, open-cell bone material on the other hand, would provide satisfactory results. Cuttings of porous metallic or biocompatible, open-cell materials with factor XIII have not previously been employed as bone substitute material.

The invention therefore relates to a bone substitute consisting of a porous metallic or a biocompatible, open-cell material which is wholly or partly impregnated with a solution comprising factor XIII, or at least some of its pores are filled with a solution comprising factor XIII.

It is intended preferably that the bone material consists of a biocompatible, open-cell substance which is at least partly bioabsorbable. Hydroxyapatite and tricalcium phosphate have very particularly proved suitable for this purpose. However, it is also possible to employ endogenous bone substance or tricoralite. Porous metallic materials have the advantage, because of their great strength, of conferring great stability on the bone.

The bone substitute material is to have a porosity of at least 25%, preferably of at least 35%, and more than 50% of the pores should have a diameter in the range from 200 to 500 microns. A biosubstitute material in which the channels connecting the individual pores of a diameter in the range from 10 to 300 microns, preferably 200 to 400 microns, is particularly suitable.

The factor XIII can be added to the bone substitute material in very diverse ways. It is also very suitable to use factor XIII in the form of microcapsules with protracted release of active substance, where the capsule wall consists of biodegradable synthetic materials, e.g. polylactic acid, or proteins.

The great advantage of the bone substitute of the invention compared with the use of a factor XIII-free, porous metallic or absorbable, open-cell material with which the factor XIII is administered to the patient in parenteral form is that particularly high factor XIII concentrations on the bone to be treated are ensured with the bone substitute material of the invention. This creates particularly good conditions for rapid and effective bone regeneration.

Bone regeneration can be further expedited by adding to the bone substitute of the invention also additional cells from autologous bone marrow or other bone-forming cells of the patient or of cells obtained from his periosteum.

The success of bone regeneration can also be promoted through the use of osteoinductive and/or osteogenic substances which are employed in addition to the factor XIII and the cells obtained from autologous bone marrow. Substances particularly suitable for this purpose are the following, which are preferably administered in the form of a suspension:
a) synthetic growth factors,
b) recombinant growth factors, preferably β growth factor (TGF-β) or FGF-2 (fibroblast growth factor);
c) natural or synthetic peptides;
d) platelet-derived growth factor (PDGF);
e) insulin-like growth factor (IGF);
f) fibrin as end product of coagulation,
g) synthetic fibrin or
h) proteins of the bone morphogenetic protein family (BMP).

The suspension of the osteoinductive or osteogenic substances can be administered in a suspension which is tolerated by the body, preferably in an aqueous suspension.

The bone substitute of the invention can be produced in various ways. In general, the porous metallic or biocompatible, open-cell bone material will be impregnated immediately before use with a solution comprising factor XIII by sucking the solution into the pores of the material by applying a vacuum. However, it is also possible to coat or to mix the bone material with a solution comprising factor XIII. In the same way, the aforementioned cells from autologous bone marrow or the cells obtained from the periosteum, and the aforementioned osteoinductive and/or osteogenic substances can be brought into contact with the bone substitute. The only decisive point is that intensive wetting of the outer and inner surface of the porous bone substitute material is achieved.

The amount of factor XIII to be introduced into the injured bone together with the porous metallic or the biocompatible, open-cell material has to date generally been from 10 to 50 units/kg of body weight on intravenous administration. However, if the bone substitute material is impregnated according to the invention with a factor XIII solution, or its pores at least partly filled with a solution comprising factor XIII, then from 0.05 to 10 units of factor XIII/kg of body weight are sufficient.

On use of the described method, the development of a granular, low-fiber, cell- and vessel-rich connective tissue, the granulation tissue, is observed after only a few days. Various cells then start to construct a cartilaginous matrix in this tissue. This process proceeds until the entire granulation tissue is replaced by cartilage and later calcified.

Use of the porous metallic or biocompatible, open-cell bone substitute material enriched according to the invention with factor XIII very considerably expedites bone regeneration. Bone regeneration in vivo can in this way be shortened by up to 40%.

1. A bone substitute consisting of a porous metallic or biocompatible, open-cell material, which is wholly or partly impregnated with a solution comprising factor XIII.

2. The bone substitute as claimed in claim 1, wherein the biocompatible, and open-cell material is at least partly bioadsorbable.

3. The bone substitute as claimed in claim 1, which has a porosity of at least 25%.

4. The bone substitute as claimed in claim 1, wherein more than 50% of the pores have a diameter in the range from 200 to 500 microns.

5. The bone substitute as claimed in claim 1, wherein the channels connecting the individual pores have a diameter in the range from 10 to 400 microns.

6. The bone substitute as claimed in claim 1, which additionally comprises cells from autologous bone marrow.

7. The bone substitute as claimed in claim 1, which additionally comprises cells obtained from the periosteum.

8. The bone substitute as claimed in claim 2, wherein the biocompatible open-cell material is hydroxyapatite.

9. The bone substitute as claimed in claim 2, wherein the biocompatible open-cell material is tricalcium phosphate.

10. The bone substitute as claimed in claim 3 wherein the porosity is at least 35%.

11. The bone substitute of claim 5 wherein the channels connecting the individual pores have a diameter of 200 to 300 microns.
